(19) United States
(12) Patent Application Publication
(10) Pub. No.: US 2010/0268473 A1
(43) Pub. Date: Oct. 21, 2010

(54) METHODS, DEVICES, AND COMPOSITIONS FOR THE HIGHLY-SENSITIVE DETECTION AND IDENTIFICATION OF DIVERSE MOLECULAR ENTITIES

(76) Inventors: Ralph A. Tripp, Watkinsville, GA (US); Richard A. Dluhy, Athens, GA (US); Yiping Zhao, Statham, GA (US); Jeremy Driskell, Athens, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/742,987

(22) PCT Filed: Feb. 19, 2009
(86) PCT No.: PCT/US2009/034488

§ 371 Date: May 14, 2010

Related U.S. Application Data
(60) Provisional application No. 61/029,680, filed on Feb. 19, 2008.

(51) Int. Cl. G01N 33/48; G01J 3/44; G06F 17/18

(52) U.S. Cl. 702/19; 356/301

(57) ABSTRACT

Embodiments of the present disclosure include a method for analysis of individual components in a multicomponent sample where the identity of the individual components is an indicator for disease.

### CROSS-REFERENCE TO RELATED APPLICATION

This application claims priority to co-pending U.S. provisional application entitled “Methods, Devices and Compositions for the Highly Sensitive Detection and Identification of Diverse Molecular Entities,” having Ser. No. 61/029,680, filed Feb. 19, 2008, which is entirely incorporated herein by reference.

### STATEMENT REGARDING FEDERALLY SPONSORED RESEARCH OR DEVELOPMENT

Aspects of this disclosure may have been made with government support under W911 NF-07-2-0065 awarded by the U.S. Army Research Laboratory. The government may have certain rights in the invention(s).

### SEQUENCE LISTING

The present disclosure includes a sequence listing incorporated herein by reference in its entirety.

### BACKGROUND

MicroRNAs (miRNAs) are small endogenous RNA molecules (19-25 nt) that regulate gene expression by targeting one or more mRNAs for translational repression or cleavage, and have been shown to have different expression profiles in various pathological conditions. Most notably, miRNAs have been associated with the development of certain types of cancer, but a growing body of evidence shows that miRNAs function to regulate virus replication following infection. Thus, miRNA expression profiles provide diagnostic and/or prognostic biomarkers of disease. Understanding the interface between miRNA expression and disease is also important to provide insights into mechanisms of disease pathogenesis and may provide novel disease intervention strategies.

The small size of miRNAs presents a significant challenge for detection. Conventional methodologies include PCR, northern blots, and microarrays where each method relies on hybridization of target RNA with a complementary probe (or oligonucleotide). In the case of miRNAs, not only is the risk of cross-hybridization high due to their short lengths, but miRNA detection probes must be labeled with a signal transducer, e.g., fluorophore which may inhibit hybridization. Northern blot detection of miRNAs, although a traditional method for detection, is labor and time intensive and requires a labeled probe to hybridize for detection. Moreover, this method requires relatively high concentrations of specimen (10-30 μg), and has a low threshold of detection, making fine specificity detection of miRNAs difficult. Quantitative reverse transcription PCR (qRT-PCR) offers the advantage of increased sensitivity of miRNA detection; however, primer selection is hindered by the short size of miRNAs. Thus, qRT-PCR is better suited to detect miRNA precursors having longer sequences than mature miRNA. Unfortunately, it has been found that levels of pre-miRNA do not always correlate with mature miRNA levels. Protocols have been developed to attach artificial tails to mature miRNA for amplification, but these require additional costly and lengthy steps.

Microarray methods offer significant improvements in sample throughput by analyzing multiple miRNAs simultaneously. However, detection of miRNAs typically requires fluorescently labeled oligonucleotides for complimentary hybridization to potential miRNAs, thus the same challenges exist as for northern blotting and PCR methods. While the throughput is high, the analysis is still labor intensive, and false-positive detection is not uncommon. Perhaps the greatest complication with this methodology is the lack of standardized protocols for consistent hybridization efficiency via removal of unhybridized sequences, as well as signal interpretation and validation.

The difficulties in miRNA detection have driven the search for new methods for miRNA detection that overcome the limitations associated with conventional methods. Gold nanoparticles and quantum dots have been incorporated into hybridization assays in place of fluorophores to successfully improve assay sensitivity. Molecular beacon approaches have been used to differentiate between single-base mismatches between miRNAs and significantly reduce the specimen concentration required for detection. Bead-based flow cytometry and RAKE adaptation of microarray technology are two promising and novel approaches which appear to reduce assay time and improve assay specificity, respectively. However, central to each of these emerging techniques is the requirement for a hybridization step. A detection method that circumvents the hybridization step would have significant impact on the accuracy, analysis time, and cost of miRNA detection.

### SUMMARY

Embodiments of the present disclosure include a method for analysis of individual and distinct components in a multicomponent sample where the identity of the individual components is an indicator for disease. In an embodiment, the individual components include individual and distinct miRNA or nucleotide sequences.

Briefly described, embodiments of the present disclosure include methods for analysis of individual and distinct components in a multicomponent sample, comprising: applying the multicomponent sample to a surface enhanced Raman spectroscopy (SERS) platform; obtaining a unique SERS spectrum for each component of the multicomponent sample; analyzing the unique SERS spectrum of each component of the multicomponent sample; and determining disease based on an identity of at least one individual component or family of components.

Briefly described, embodiments of the present disclosure include a method for identification, differentiation, and/or quantification of individual and distinct components in a multicomponent sample, comprising: applying the multicomponent sample to a surface enhanced Raman spectroscopy (SERS) platform; obtaining a unique SERS spectrum for each component of the multicomponent sample; and analyzing the unique SERS spectrum of each component of the multicomponent sample.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a graph that illustrates the average SERS spectra for let-7a (2M1), miR-133a (2M10), and a mixture of 0.6 μg let-7a and 0.4 μg miR-133a (2M5). Average spectra collected from 3 substrates are presented to highlight spectral reproducibility. All spectra have been baseline corrected and unit vector normalized.

FIG. 2 is a graph that illustrates the average SERS spectra for mixtures of let-7a and miR-133a: 2M1=1.0 μg let-7a, 2M4=0.8 μg let-7a and 0.2 μg miR-133a, 2M5=0.6 μg let-7a and 0.4 μg miR-133a, 2M6=0.4 μg let-7a and 0.6 μg miR-133a, 2M7=0.2 μg let-7a and 0.8 μg miR-133a, and 2M10=1.0 μg miR-133a. All spectra have been baseline corrected and unit vector normalized.

FIGS. 3A through 3D are graphs that illustrate PLS results for 2-component mixtures of let-7a and miR-133a (FIGS. 3A and 3B) cross-validation predictions for calibration model and (FIGS. 3C and 3D) predictions for external validation. The solid line is a plot of x=y, to serve as a guide.

FIG. 4 illustrates a ternary plot illustrating the composition of 3-component mixtures of let-7a, miR-133a, and miR-16.

FIGS. 5A through 5B are graphs that illustrate a plot of PLS regression cross-validation predicted versus true concentrations of (FIG. 5A) let-7a, (FIG. 5B) miR-133a, and (FIG. 5C) miR-16 for 3-component mixtures. The solid line is a plot of x=y, to serve as a guide.

FIGS. 6A through 6B are graphs that illustrate PLS predictions for let-7a in the presence of four other miRNA sequences (FIG. 6A) cross-validation predictions for calibration model and (FIG. 6B) predictions for external validation. The solid line is a plot of x=y, to serve as a guide.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

Use of the phrase “peptides”, “polypeptide”, or “protein” is intended to encompass a protein, a glycoprotein, a polypeptide, a peptide, fragments thereof and the like, whether isolated from nature, of viral, bacterial, plant, or animal (e.g., mammalian, such as human) origin, or synthetic, and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

Use of the term “nucleotide” is intended to encompass molecules which comprise the structural units of RNA and DNA. A nucleotide is composed of a nitrogenous base and a five-carbon sugar (either ribose or 2′-deoxyribose), and one to three phosphate groups. A nucleobase and sugar comprise a nucleoside. Cyclic nucleotides are a form comprised of a phosphate group bound to two of the sugar's hydroxyl groups. Ribonucleotides are nucleotides where the sugar is ribose, and deoxyribonucleotides contain the sugar deoxyribose. Nucleotides can contain either a purine or pyrimidine base.

Use of the term “polynucleotide” is intended to encompass DNA, RNA, and miRNA whether isolated from nature, of viral, bacterial, plant or animal (e.g., mammalian, such as human) origin, or synthetic; whether single-stranded or double-stranded; or whether including naturally or non-naturally occurring nucleotides, or chemically modified. As used herein, “polynucleotides” include single or multiple stranded configurations, where one or more of the strands may or may not be completely aligned with another. The terms “polynucleotide” and “oligonucleotide” shall be generic to polydeoxynucleotides (containing 2-deoxy-D-ribose), to polyribonucleotides (containing D-ribose), to any other type of polynucleotide which is an N-glycoside of a purine or pyrimidine base, and to other polymers in which the conventional backbone has been replaced with a non-naturally occurring or synthetic backbone or in which one or more of the conventional bases has been replaced with a non-naturally occurring or synthetic base. An “oligonucleotide” generally refers to a nucleotide multimer of about 2 to 100 nucleotides in length, while a “polynucleotide” includes a nucleotide multimer having any number of nucleotides greater than 1, although they are often used interchangeably.

Use of the term “affinity” can include biological interactions and/or chemical interactions. The biological interactions can include, but are not limited to, bonding or hybridization among one or more biological functional groups located on the first biomolecule and the second biomolecule. In this regard, the first (or second) biomolecule can include one or more biological functional groups that selectively interact with one or more biological functional groups of the second (or first) biomolecule. The chemical interaction can include, but is not limited to, bonding among one or more functional groups (e.g., organic and/or inorganic functional groups) located on the biomolecules.

### DISCUSSION

Embodiments of the present disclosure include methods for identification, differentiation, and/or quantification of individual components in a multicomponent sample. Embodiments of the present disclosure include methods for quantification of individual and distinct components in a multicomponent sample where the individual components include individual microRNA (miRNA) or nucleotide sequences. In an embodiment, the method includes a rapid, sensitive, and quantitative method for identification of individual and distinct miRNA or nucleotide sequences in multicomponent samples using surface enhanced Raman spectroscopy (SERS). Embodiments of the present disclosure include methods where individual miRNA or nucleotide sequences can be detected in about 10-30 seconds. In addition, embodiments of the present disclosure can be used in miRNA profiling, which is described in detail in Example 1.

Embodiments of the present disclosure include a method for analysis of individual and distinct components in a multicomponent sample. The method includes applying the multicomponent sample to a surface enhanced Raman spectroscopy (SERS) platform. In an embodiment, application of the multicomponent sample to a SERS platform includes spotting the sample onto the prepared SERS substrate and allowing it to dry at room temperature. Next, a unique SERS spectrum is obtained for each component of the multicomponent sample. Subsequently, the unique SERS spectrum of each component of the multicomponent sample is statistically analyzed. Then, a disease or condition can be determined based on an identity of at least one individual component (e.g., cancer, cardiac disease). In an embodiment, the analysis includes identification, differentiation, and/or quantification of the individual and distinct components of the multicomponent sample. In another embodiment, the analysis includes the quantification of miRNA sequences in a multicomponent sample.

The unique SERS spectrum of a single component in the multicomponent sample is independent of the number of components in the sample. Furthermore, the only change in the unique SERS spectrum of the individual components is that the intensity of the signature changes with concentration.

As described herein, a multicomponent sample can include a sample that contains at least a mixture of different miRNA or nucleotide sequences. The miRNA sequences may be miRNA genes that are first transcribed as long pri-miRNAs, processed pre-miRNAs of ˜70 nt precursors (pre-miRNA) having stem-loop structures, or mature miRNAs of ˜22 nt. The miRNA sequences of mature miRNA may contain seed sequence or mutations affecting its expression and regulation of its target gene(s).

Embodiments of the present disclosure can include a multicomponent sample concentration that is dilute. In an embodiment, the multicomponent sample concentration is about 0.04 to 1.0 μg/μL or about 0.0 to 1.0 μg/μL for each component in the sample. In another embodiment, the concentration is about 0.04 to 1.0 μg/μL for each miRNA in the sample. Embodiments of the present disclosure include a multicomponent sample concentration where the total miRNA concentration is about 1.0 μg/μL.

Embodiments of the present disclosure include multicomponent samples selected from the group consisting of: blood, saliva, tears, phlegm, sweat, urine, plasma, lymph, spinal fluid, cells, microorganisms, a combination thereof, and aqueous dilutions thereof.

As described herein, the analysis of the SERS spectra can include using regression analysis (e.g., partial least squares (PLS) regression analysis or classical least squares (CLS)) of the SERS spectra to determine the concentration of each component in the multicomponent sample. A unique SERS spectrum includes the SERS spectrum uniquely characteristic for each component. Where the component is an individual miRNA sequence, the unique SERS spectrum includes the SERS spectrum uniquely characteristic for the miRNA sequence. In addition, embodiments of the present disclosure provide the ability to distinguish between or among the unique SERS spectrum for each individual miRNA in a sample. The term “distinguish” refers to the ability to separately identify each of the miRNA in a sample and/or SERS spectrum even when the sample includes multiple miRNA.

As described herein, quantification includes determining the concentration of the individual components within the multicomponent sample. The signatures of the individual components in the multicomponent sample change in intensity with concentration.

Embodiments of the present disclosure include determination of a disease or condition (e.g., cancer, cardiac disease) based on the identity of at least one individual component or family of components of the multicomponent sample. A family of components refers to the handful of miRNAs that are used for diagnosis of disease. Many times, it is not one miRNA that has diagnostic value, but the concentration of several miRNAs that has diagnostic value. Alternatively, a family of miRNAs can refer to a number of closely related miRNAs (e.g., the let-7 family consists of let-7a, let-7b, let-7c, let-7d . . . let-7i).

Diseases or conditions that may be identified can include solid organ and hematological malignancies, heart disease, immune response elements, organ development, neurodegenerative diseases, and susceptibility to disease. Embodiments of the present disclosure include the detection of an individual miRNA sequence where the detection is an indicator for the detection of cancer.

Due to its prevalence, and the potential impact of discovering a diagnostic or prognostic indicator for this disease, lung cancer has received much attention with respect to miRNA expression analysis. Comparative miRNA profiles of normal versus various lung cancer type tissues have lead to several important findings. First, independent research groups have identified differentially expressed miRNAs between cancerous and corresponding normal lung tissue that can serve as diagnostic biomarkers (Jay, C.; Nemunaitis, J.; Chen, P.; Fulgham, P.; Tong, A. W. DNA and Cell Biology 2007, 26, 293-300, which is herein incorporated by reference for the corresponding discussion). Second, studies have also found that many of the differentially expressed miRNAs have prognostic value. For example, independent laboratories have reported that high expression levels of miR-155 or miR-21 or low expression of let-7 are indicators of poor survival (Markou, A.; Tsaroucha, E. G.; Kaklamanis, L.; Fotinou, M.; Georgoulias, V.; Lianidou, E. S. Clinical Chemistry 2008, 54, 1696-1704; Yanaihara, N.; Caplen, N.; Bowman, E.; Seike, M.; Kumamoto, K.; Yi, M.; Stephens, R. M.; Okamoto, A.; Yokota, J.; Tanaka, T.; Colin, G. A.; Liu, C. G.; Croce, C. M.; Harris, C. C. Cancer Cell 2006, 9, 189-198. which are herein incorporated by reference for the corresponding discussion). Third, miRNAs may have therapeutic value. Transfection of cancerous cells with let-7 mimics has been shown to reduce lung cancer proliferation; an effect that has been replicated both in vitro and in vivo (Johnson, C. D.; Esquela-Kerscher, A.; Stefani, G.; Byrom, M.; Kelnar, K.; Ovcharenko, D.; Wilson, M.; Wang, X.; Shelton, J.; Shingara, J.; Chin, L.; Brown, D.; Slack, F. J. Cancer Res 2007, 67, 7713-7722; Kumar, M. S.; Erkeland, S. J.; Pester, R. E.; Chen, C. Y.; Ebert, M. S.; Sharp, P. A.; Jacks, T. Proc Natl Acad Sci USA 2008, 105, 3903-3908; Takamizawa, J.; Konishi, H.; Yanagisawa, K.; Tomida, S.; Osada, H.; Endoh, H.; Harano, T.; Yatabe, Y.; Nagino, M.; Nimura, Y.; Mitsudomi, T.; Takahashi, T. Cancer Research 2004, 64, 3753-3756, which are herein incorporated by reference for the corresponding discussion). Evidence suggests that routine miRNA profiling could facilitate cancer diagnosis, prognosis, and determine appropriate treatments.

Embodiments of the present disclosure include a method for analysis of individual and distinct components in a multicomponent sample where the individual components comprise individual and distinct miRNA or nucleotide sequences. In an embodiment, the method includes a detection method that circumvents the hybridization step of conventional methodologies, which has a significant impact on the accuracy, analysis time, and cost of miRNA detection. Thus, embodiments of the present disclosure are advantageous over current techniques.

In an embodiment of the present disclosure, the SERS platform includes a Ag nanorod array substrate. In another embodiment, the Ag nanorod array substrate is prepared by oblique angle vapor deposition (OAD). Embodiments of the present disclosure include Ag nanorod array substrates comprising individual nanorods with a length of about 850 to 950 nm (e.g., 900 nm).

Embodiments of the present disclosure include SERS substrates where the nanorods are selected from one of the following materials: a metal, a metal oxide, a metal nitride, a metal oxynitride, a polymer, a multicomponent material, and combinations thereof. In an embodiment, the material is selected from one of the following: silver, nickel, aluminum, silicon, gold, platinum, palladium, titanium, cobalt, copper, zinc, oxides of each, nitrides of each, oxynitrides of each, carbides of each, and combinations thereof.

Embodiments of the present disclosure include a method for identification, differentiation, and/or quantification of individual components in a multicomponent sample. In an embodiment, the method includes applying the multicomponent sample to a surface enhanced Raman spectroscopy (SERS) platform. Next, a unique SERS spectrum for each component of the multicomponent sample can be obtained. In an embodiment, the individual components of the multicomponent sample comprise individual miRNA or nucleotide sequences. Subsequently, the unique SERS spectrum of each component of the multicomponent sample can be analyzed.

Embodiments of the present disclosure include a method for identification, differentiation, and/or quantification of individual and distinct components in a multicomponent sample where the SERS platform comprises a Ag nanorod array substrate. In an embodiment, the Ag nanorod array substrate is prepared by oblique angle vapor deposition (OAD).

Embodiments of the present disclosure include a multicomponent sample comprising at least two components. Embodiments of the present disclosure include a multicomponent sample comprising about three components. Embodiments of the present disclosure include a multicomponent sample comprising about five components. In an embodiment, the components (e.g., two, three, four, or five components) are miRNA selected from the group consisting of: hsa-let-7a, hsa-miR-133a, hsa-miR-21, hsa-miR-16, and hsa-miR-24a. These components are representative of miRNA families that have been linked to human disease.

### EXAMPLES

### Example 1

### Introduction

MicroRNAs (miRNAs) are small endogenous RNA molecules (−21-25 nt) that regulate gene expression by targeting one or more mRNAs for translational repression or cleavage (Bartel, D. P. Cell 2004, 116, 281-297; Scherr, M.; Eder, M. Curr. Opin. Mol. Ther. 2004, 6, 129-135; Zhang, B.; Wang, Q.; Pan, X. J. Cell Physiol. 2007, 210, 279-289, which are herein incorporated by reference for the corresponding discussion), and have been shown to have different expression profiles in various pathological conditions. Most notably, miRNAs have been associated with the development of certain types of cancer (Calin, G. A.; Croce, C. M. Semin. Oncol. 2006, 33, 167-173; Calin, G. A.; Croce, C. M. Cancer Res. 2006, 66, 7390-7394; Cimmino, A.; Calin, G. A.; Fabbri, M.; Iorio, M. V.; Ferracin, M.; Shimizu, M.; Wojcik, S. E.; Aqeilan, R. I.; Zupo, S.; Dono, M.; Rassenti, L.; Alder, H.; Volinia, S.; Liu, C. G.; Kipps, T. J.; Negrini, M.; Croce, C. M. Proc. Natl. Acad. Sci. USA 2005, 102, 13944-13949; Hammond, S. M. Cancer Chemother. Pharmacol. 2006, 58 Suppl 1, s63-68; He, L.; Thomson, J. M.; Hemann, M. T.; Hernando-Monge, E.; Mu, D.; Goodson, S.; Powers, S.; Cordon-Cardo, C.; Lowe, S. W.; Hannon, G. J.; Hammond, S. M. Nature 2005, 435, 828-833; Michael, M. Z.; SM, O. C.; van Holst Pellekaan, N. G.; Young, G. P.; James, R. J. Mol. Cancer Res. 2003, 1, 882-891; Tagawa, H.; Seto, M. Leukemia 2005, 19, 2013-2016, which are herein incorporated by reference for the corresponding discussion), but a growing body of evidence shows that miRNAs function to regulate virus replication following infection (Jopling, C. L.; Yi, M.; Lancaster, A. M.; Lemon, S. M.; Sarnow, P. Science 2005, 309, 1577-1581; Lecellier, C.-H.; Dunoyer, P.; Arar, K.; Lehmann-Che, J.; Eyquem, S.; Himber, C.; Saib, A.; Voinnet, O. Science 2005, 308, 557-560; Bennasser, Y.; Le, S. Y.; Yeung, M. L.; Jeang, K. T. Retrovirology 2004, 1, 43; Cullen, B. R. Nat. Genet. 2006, 38 Suppl, S25-30; Pfeffer, S.; Sewer, A.; Lagos-Quintana, M.; Sheridan, R.; Sander, C.; Grasser, F. A.; van Dyk, L. F.; Ho, C. K.; Shuman, S.; Chien, M.; Russo, J. J.; Ju, J.; Randall, G.; Lindenbach, B. D.; Rice, C. M.; Simon, V.; Ho, D. D.; Zavolan, M.; Tuschl, T. Nat. Methods 2005, 2, 269-276; Pfeffer, S.; Voinnet, O. Oncogene 2006, 25, 6211-6219, which are herein incorporated by reference for the corresponding discussion). Thus, miRNA expression profiles provide diagnostic and/or prognostic biomarkers of disease. Understanding the interface between miRNA expression and disease is also important to provide insights into mechanisms of disease pathogenesis and may provide novel disease intervention strategies.

The small size of miRNAs presents a significant challenge for detection. Conventional methodologies include PCR, northern blots, and microarrays where each method relies on hybridization of target RNA with a complementary probe (or oligonucleotide). In the case of miRNAs, not only is the risk of cross-hybridization high due to their short lengths, but miRNA detection probes must be labeled with a signal transducer, e.g., fluorophore which may inhibit hybridization. Northern blot detection of miRNAs, although a traditional method for detection (Lu, J.; Getz, G.; Miska, E. A.; Alvarez-Saavedra, E.; Lamb, J.; Peck, D.; Sweet-Cordero, A.; Ebert, B. L.; Mak, R. H.; Ferrando, A. A.; Downing, J. R.; Jacks, T.; Horvitz, H. R.; Golub, T. R. Nature 2005, 435, 834-838, which is incorporated by reference for the corresponding discussion), is labor and time intensive and requires a labeled probe to hybridize for detection. Moreover, this method requires relatively high concentrations of specimen (10-30 μg), and has a low threshold of detection, making fine specificity detection of miRNAs difficult (Cissell, K. A.; Shrestha, S.; Deo, S. K. Anal. Chem. 2007, 79, 4754-4761, which is herein incorporated by reference for the corresponding discussion). Quantitative reverse transcription PCR (qRT-PCR) offers the advantage of increased sensitivity of miRNA detection; however, primer selection is hindered by the short size of miRNAs. Thus, qRT-PCR is better suited to detect miRNA precursors having longer sequences than mature miRNA. Unfortunately, it has been found that levels of pre-miRNA do not always correlate with mature miRNA levels. Protocols have been developed to attach artificial tails to mature miRNA for amplification (Chen, C. F.; Ridzon, D. A.; Broomer, A. J.; Zhou, Z. H.; Lee, D. H.; Nguyen, J. T.; Barbisin, M.; Xu, N. L.; Mahuvakar, V. R.; Andersen, M. R.; Lao, K. Q.; Livak, K. J.; Guegler, K. J. Nucleic Acids Res. 2005, 33; Shi, R.; Chiang, V. L. Biotechniques 2005, 39, 519-525, which are herein incorporated by reference for the corresponding discussion), but these require additional costly and lengthy steps.

Microarray methods offer significant improvements in sample throughput by analyzing multiple miRNAs simultaneously (Barad, O.; Meiri, E.; Avniel, A.; Aharonov, R.; Barzilai, A.; Bentwich, I.; Einav, U.; Glad, S.; Hurban, P.; Karov, Y.; Lobenhofer, E. K.; Sharon, E.; Shiboleth, Y. M.; Shtutman, M.; Bentwich, Z.; Einat, P. Genome Res. 2004, 14, 2486-2494; Liu, C.-G.; Calin, G. A.; Meloon, B.; Gamliel, N.; Sevignani, C.; Ferracin, M.; Dumitru, C. D.; Shimizu, M.; Zupo, S.; Dono, M.; Alder, H.; Bullrich, F.; Negrini, M.; Croce, C. M. Proc. Natl. Acad. Sci. USA 2004, 101, 9740-9744; Nelson, P. T.; Baldwin, D. A.; Scearce, L. M.; Oberholtzer, J. C.; Tobias, J. W.; Mourelatos, Z. Nat. Methods 2004, 1, 155-161; Thomson, J. M.; Parker, J. S.; Hammond, S. M. In Methods in Enzymology; Rossi, J. J., Hannon, G. J., Eds.; Academic Press San Diego, Calif., 2007; Vol. Volume 427, pp 107-122; Yan, N. H.; Lu, Y. L.; Sun, H. Q.; Tao, D. C.; Zhang, S. Z.; Liu, W. Y.; Ma, Y. X. Reproduction 2007, 134, 73-79; Yin, J. Q.; Zhao, R. C. Methods 2007, 43, 123-130, which are herein incorporated by reference for the corresponding discussion). However, detection of miRNAs typically require fluorescently labeled oligonucleotides for complimentary hybridization to potential miRNAs, thus the same challenges exist as for northern blotting and PCR methods. While the throughput is high, the analysis is still labor intensive, and false-positive detection is not uncommon. Perhaps the greatest complication with this methodology is the lack of standardized protocols for consistent hybridization efficiency via removal of unhybridized sequences, as well as signal interpretation and validation.

The difficulties in miRNA detection have driven the search for new methods for miRNA detection that overcome the limitations associated with conventional methods. Gold nanoparticles and quantum dots have been incorporated into hybridization assays in place of fluorophores to successfully improve assay sensitivity (Liang, R. Q.; Li, W.; Li, Y.; Tan, C. Y.; Li, J. X.; Jin, Y. X.; Ruan, K. C. Nucleic Acids Res. 2005, 33, which is herein incorporated by reference for the corresponding discussion). Molecular beacon approaches have been used to differentiate between single-base mismatches between miRNAs and significantly reduce the specimen concentration required for detection (Hartig, J. S.; Grune, I.; Najafi-Shoushtari, S. H.; Famulok, M. Journal of the American Chemical Society 2004, 126, 722-723, which is herein incorporated by reference for the corresponding discussion). Bead-based flow cytometry and RAKE adaptation of microarray technology are two promising and novel approaches which appear to reduce assay time and improve assay specificity, respectively (Lu, J.; Getz, G.; Miska, E. A.; Alvarez-Saavedra, E.; Lamb, J.; Peck, D.; Sweet-Cordero, A.; Ebert, B. L.; Mak, R. H.; Ferrando, A. A.; Downing, J. R.; Jacks, T.; Horvitz, H. R.; Golub, T. R. Nature 2005, 435, 834-838; Nelson, P. T.; Baldwin, D. A.; Scearce, L. M.; Oberholtzer, J. C.; Tobias, J. W.; Mourelatos, Z. Nat. Methods 2004, 1, 155-161, which are herein incorporated by reference for the corresponding discussion). However, central to each of these emerging techniques is the requirement for a hybridization step. A detection method that circumvents the hybridization step would have significant impact on the accuracy, analysis time, and cost of miRNA detection.

Recently, we demonstrated that surface enhanced Raman spectroscopy (SERS) may be used as a label-free spectroscopic method for detecting individual miRNA sequences, including single base mismatches (Driskell, J. D.; Seto, A. G.; Jones, L. P.; Jokela, S.; Dluhy, R. A.; Zhao, Y. P.; Tripp, R. A. Biosens. Bioelectron, which is herein incorporated by reference for the corresponding discussion). SERS is a spectroscopic technique in which the analyte is adsorbed onto a nanometrically roughened metal surface that serves as a platform to enhance the Raman scattered signal by up to 14 orders of magnitude (Willets, K.; Duyne, R. P. V. Ann. Rev. Phys. Chem 2007, 58, 267-297; Stiles, P. L.; Dieringer, J. A.; Shah, N. C.; Van Duyne, R. P. Ann. Rev. Anal. Chem 2008, 1, 601-626, which are herein incorporated by reference for the corresponding discussion). Our laboratories have established that Ag nanorod arrays fabricated by an oblique angle deposition method produce highly sensitive and reproducible SERS substrates with enhancements >10⁸ (Chaney, S. B.; Shanmukh, S.; Zhao, Y.-P.; Dluhy, R. A. Appl. Phys. Lett. 2005, 87, 31908-31910; Driskell, J. D.; Shanmukh, S.; Chaney, S. B.; Tang, X.-J.; Zhao, Y.-P.; Dluhy, R. A. J. Phys. Chem. C 2008, 112, 895-901, which are herein incorporated by reference for the corresponding discussion). SERS has previously been employed in the study of nucleic acids, with much of the previous work devoted to the analysis of DNA and RNA structure (Green, M.; Liu, F. M.; Cohen, L.; Kollensperger, P.; Cass, T. Faraday Discuss. 2006, 132, 269-280; Kattumuri, V.; Chandrasekhar, M.; Guha, S.; Raghuraman, K.; Katti, K. V.; Ghosh, K.; Patel, R. J. Appl. Phys. Lett. 2006, 88; Kneipp, K.; Flemming, J. J. Mol. Struct. 1986, 145, 173-179; Koglin, E.; Sequaris, J. M.; Valenta, P. J. Mol. Struct. 1982, 79, 185-189; Nabiev, I. R.; Sokolov, K. V.; Voloshin, O. N. J. Raman Spectrosc. 1990, 21, 333-336; Otto, C.; Tweel, T. J. J. v.; deMul, F. F. M.; Greve, J. J. Raman Spectrosc. 1986, 17; Thornton, J.; Force, R. K. Appl. Spectrosc. 1991, 45, 1522-1526; Suh, J. S.; Moskovits, M. J. Am. Chem. Soc. 1986, 108, 4711-4718, which are herein incorporated by reference for the corresponding discussion). However, our recent article was the first demonstration that Ag nanorod-based SERS is sufficiently sensitive to identify the molecular spectra of individual miRNA sequences (Driskell, J. D.; Seto, A. G.; Jones, L. P.; Jokela, S.; Dluhy, R. A.; Zhao, Y. P.; Tripp, R. A. Biosens. Bioelectron, which is herein incorporated by reference for the corresponding discussion).

Our previously published study demonstrated that SERS was a sensitive, label-free method for identification of synthetic miRNAs in single-component samples. The studies described in this report demonstrate that SERS is not only able to identify, but is also able to accurately and quantitatively determine the concentrations of, individual miRNA sequences within multicomponent mixtures of miRNA. Two-, three-, and five-component mixtures of miRNAs were prepared with varying concentrations of each component. Partial least-squares (PLS) analysis of the SERS spectra is shown to provide accurate determination of concentrations for each component. Extension of the methodology developed in this report to miRNA profiling of total RNA samples extracted from cells and/or tissue is discussed.

### Experimental Section

miRNA Samples. Five human miRNAs were synthesized and graciously provided as dehydrated samples by Thermo Fisher Scientific, Dharmacon (Table 1): hsa-miR-21 (SEQ. ID No. 2), hsa-let-7a (SEQ. ID No. 5), hsa-miR-16 (SEQ. ID No. 1), hsa-miR-24a (SEQ. ID No. 3), and hsa-miR-133a (SEQ. ID No. 4). MiRNAs were selected from Sanger miRBase release version 9.0. Each miRNA was resuspended in RNase-free Milli-Q water at a concentration of 1 μg/μL. Sequence details are given in Table 1.

TABLE 1
miRNA sequences.

| miRNA | Sequence | |
|---|---|---|
| miR-16 | U.A.G.C.A.G.C.A.C.G.U.A.A.A.U.A.U.U.G.G.C.G | (SEQ. ID No. 1) |
| miR-21 | U.A.G.C.U.U.A.U.C.A.G.A.C.U.G.A.U.G.U.U.G.A | (SEQ. ID No. 2) |
| miR-24a | U.G.G.C.U.C.A.G.U.U.C.A.G.C.A.G.G.A.A.C.A.G | (SEQ. ID No. 3) |
| miR-133a | U.U.G.G.U.C.C.C.C.U.U.C.A.A.C.C.A.G.C.U.G.U | (SEQ. ID No. 4) |
| let-7a | U.G.A.G.G.U.A.G.U.A.G.G.U.U.G.U.A.U.A.G.U.U | (SEQ. ID No. 5) |

Initial experiments focused on two-component mixtures of hsa-let-7a and hsa-miR-133a prepared in various ratios. The total concentration in each sample was held constant at 1.00 μg/μL, but the concentration of each component was varied from 0-1.00 μg/μL. Three-component mixtures of hsa-let-7a, hsa-miR-133a, and hsa-miR-16 were then prepared for analysis. The total miRNA concentration in the three-component mixtures was held constant at 1.00 μg/μL as the relative ratios of each component were varied. A final series of experiments examined samples in which all five of the miRNAs noted above were mixed to a total concentration of 1.00 μg/μL, but the concentration of hsa-let-7a was varied. Details of each of the sample compositions are provided in Table 2.

TABLE 2
Composition of miRNA samples.

| | | let-7a | miR-133a | miR-16 | miR-21 | miR-24a |
|---|---|---|---|---|---|---|
| | Sample | μg/μL | μg/μL | μg/μL | μg/μL | μg/μL |
| 2-component | 2M1 | 1 | 0 | — | — | — |
| mixtures | 2M2 | 0.96 | 0.04 | — | — | — |
| | 2M3 | 0.9 | 0.1 | — | — | — |
| | 2M4 | 0.8 | 0.2 | — | — | — |
| | 2M5 | 0.6 | 0.4 | — | — | — |
| | 2M6 | 0.4 | 0.6 | — | — | — |
| | 2M7 | 0.2 | 0.8 | — | — | — |
| | 2M8 | 0.1 | 0.9 | — | — | — |
| | 2M9 | 0.04 | 0.96 | — | — | — |
| | 2M10 | 0 | 1 | — | — | — |
| 3-component | 3M1 | 0.6 | 0.2 | 0.2 | — | — |
| mixtures | 3M2 | 0.25 | 0.4 | 0.35 | — | — |
| | 3M3 | 0.1 | 0.7 | 0.2 | — | — |
| | 3M4 | 0 | 0.25 | 0.75 | — | — |
| | 3M5 | 1 | 0 | 0 | — | — |
| | 3M6 | 0 | 1 | 0 | — | — |
| | 3M7 | 0.2 | 0.6 | 0.2 | — | — |
| | 3M8 | 0.4 | 0.1 | 0.5 | — | — |
| | 3M9 | 0.05 | 0.8 | 0.15 | — | — |
| | 3M10 | 0.25 | 0.45 | 0.3 | — | — |
| | 3M11 | 0.8 | 0.15 | 0.05 | — | — |
| | 3M12 | 0.01 | 0.84 | 0.15 | — | — |
| | 3M13 | 0.15 | 0.01 | 0.84 | — | — |
| | 3M14 | 0.84 | 0.15 | 0.01 | — | — |
| | 3M15 | 0 | 0 | 1 | — | — |
| 5-component | 5M1 | 0.893 | 0.027 | 0.027 | 0.027 | 0.027 |
| mixtures | 5M2 | 0.714 | 0.071 | 0.071 | 0.071 | 0.071 |
| | 5M3 | 0.556 | 0.111 | 0.111 | 0.111 | 0.111 |
| | 5M4 | 0.385 | 0.154 | 0.154 | 0.154 | 0.154 |
| | 5M5 | 0.333 | 0.167 | 0.167 | 0.167 | 0.167 |
| | 5M6 | 0.273 | 0.182 | 0.182 | 0.182 | 0.182 |
| | 5M7 | 0.2 | 0.2 | 0.2 | 0.2 | 0.2 |
| | 5M8 | 0.111 | 0.222 | 0.222 | 0.222 | 0.222 |
| | 5M9 | 0.059 | 0.235 | 0.235 | 0.235 | 0.235 |
| | 5M10 | 0 | 0.25 | 0.25 | 0.25 | 0.25 |
| | 5M11 | 1 | 0 | 0 | 0 | 0 |

Silver Nanorod Array Fabrication. Silver nanorod arrays that served as enhancing substrate for SERS were prepared using the oblique angle vapor deposition (OAD) technique. The nanofabrication method has been previously described in detail (Chaney, S. B.; Shanmukh, S.; Zhao, Y.-P.; Dluhy, R. A. Appl. Phys. Lett. 2005, 87, 31908-31910; Driskell, J. D.; Shanmukh, S.; Chaney, S. B.; Tang, X.-J.; Zhao, Y.-P.; Dluhy, R. A. J. Phys. Chem. C 2008, 112, 895-901; Shanmukh, S.; Jones, L.; Driskell, J.; Zhao, Y.; Dluhy, R.; Tripp, R. Nano Lett. 2006, 6, 2630-2636, which are herein incorporated by reference for the corresponding discussion). Briefly, microscope slides were cut into 1×1 cm chips to serve as the base of the nanorod array. The glass substrates were then cleaned with hot piranha solution (80% sulfuric acid, 20% hydrogen peroxide), rinsed with DI water, dried with a stream of N₂(g), and loaded into a homemade electron-beam evaporation system (Chaney, S. B.; Shanmukh, S.; Zhao, Y.-P.; Dluhy, R. A. Appl. Phys. Lett. 2005, 87, 31908-31910; Zhao, Y.-P.; Chaney, S. B.; Shanmukh, S.; Dluhy, R. A. J. Phys. Chem. B 2006, 110, 3153-3157, which are herein incorporated by reference for the corresponding discussion). A 20-nm film of Ti was deposited onto the glass to serve as an adhesion layer, followed by a 500-nm film of Ag at a deposition rate of 0.3 nm/s. The angle of incidence was normal to the glass surface for each of these depositions to produce smooth and continuum thin films. The substrates were then rotated 86° with respect to the vapor incident direction, and Ag nanorods were grown at this oblique angle at a deposition rate of 0.3 nm/s for approximately 100 min. Each deposition step was automated using a feedback loop integrated quartz crystal microbalance to record the deposition rate and thickness, and a computer controlled power supply to adjust the electron-beam current. As reported elsewhere, these deposition conditions result in optimal SERS substrates with overall nanorod lengths of ˜900 nm (Zhao, Y.-P.; Chaney, S. B.; Shanmukh, S.; Dluhy, R. A. J. Phys. Chem. B 2006, 110, 3153-3157, which is herein incorporated by reference for the corresponding discussion).

SERS Measurements. MiRNA samples were spotted (1 μL) onto the prepared SERS substrates and allowed to dry at room temperature. A minimum of 5 spectra were recorded from different locations within each 1 μL spot to ensure representative sampling and incorporate spot-to-spot variability in signal. To account for substrate-to-substrate reproducibility, each miRNA was applied to multiple substrates (n=3-6). In total, 15-30 spectra for each sample (the pure miRNAs or their mixtures) were recorded in each experiment.

A Renishaw in Via Raman microscope system was used to acquire SERS spectra. A 785 nm near-IR diode laser was used as the excitation source, and the laser was focused into ˜115×11 μm spot using a 5× objective (N.A.=0.12). The laser power was set to 10%, where the power at the sample surface was ˜15 mW. Extended scan spectra with a spectral range of 400-1800 cm⁻¹ were acquired using a 10-s integration.

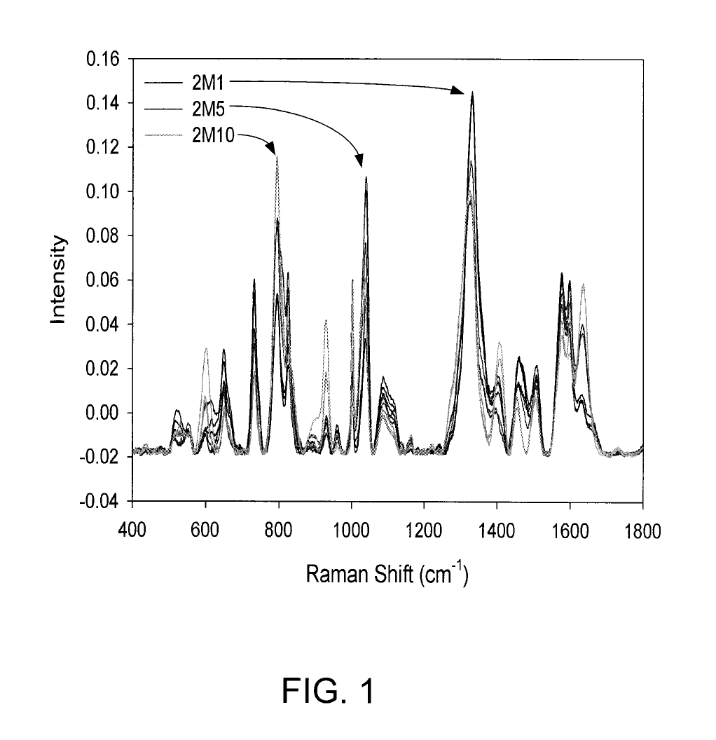

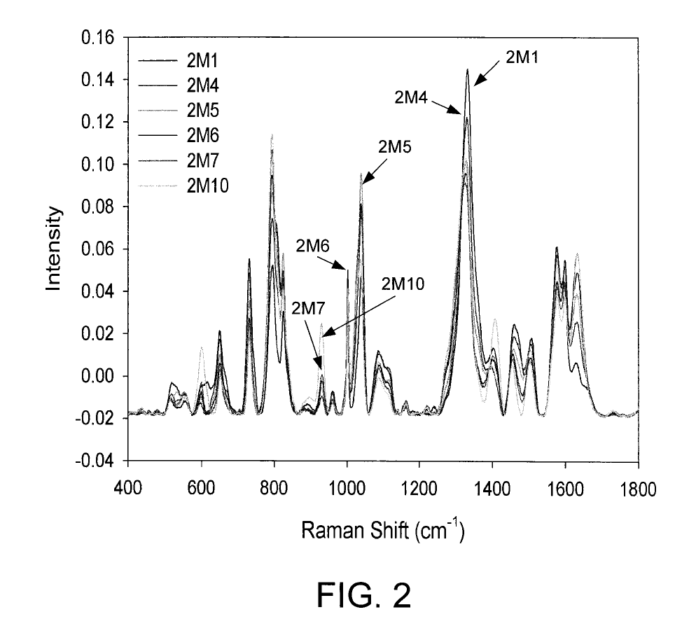

Data Analysis. Spectral reproducibility within and among substrates was crudely interrogated by visually comparing the SERS spectra. For this analysis, the spectra were baseline corrected using a concave rubber band algorithm (OPUS, Bruker Optics, Inc., Billerica, Mass.) with 10 iterations and 64 points, and then vector normalized. These steps allowed for direct comparison of Raman band locations and relative peak intensities as shown in FIGS. 1 and 2.

Partial least squares (PLS) analysis was utilized to quantify each of the miRNA sequences in the sample mixtures. Prior to PLS, the raw SERS spectra were derivatized (1^{st}-order derivative; 9-point, 2^{nd}-order polynomial Savitzky-Golay algorithm), normalized to unit vector length, and mean-centered. This pretreatment of the data eliminates complicating contributions from variations in the baseline or slight heterogeneities in the substrate enhancement factors. All preprocessing steps and the PLS analysis were performed with PLS Toolbox v4.0 (Eigen Vector Research Inc., Wenatchee, Wash.), operating in the MATLAB environment (v7.2, The Mathworks Inc., Natick, Mass.).

### Results and Discussion

Quantitative Analysis of 2-Component Mixtures. Previous studies have demonstrated that Ag nanorod substrates fabricated using oblique angle deposition (OAD) methods provided impressive spectral reproducibility for small molecules, viruses, and individual miRNA sequences (Driskell, J. D.; Shanmukh, S.; Chaney, S. B.; Tang, X.-J.; Zhao, Y.-P.; Dluhy, R. A. J. Phys. Chem. C 2008, 112, 895-901; Shanmukh, S.; Jones, L.; Driskell, J.; Zhao, Y.; Dluhy, R.; Tripp, R. Nano Lett. 2006, 6, 2630-2636; Shanmukh, S.; Jones, L.; Zhao, Y.-P.; Driskell, J.; Tripp, R. A.; Dluhy, R. A. Anal. Bioanal. Chem. 2008, 390, 1551-1555; Driskell, J. D.; Seto, A. G.; Jones, L. P.; Jokela, S.; Dluhy, R. A.; Zhao, Y. P.; Tripp, R. A. Biosens. Bioelectron, which are herein incorporated by reference for the corresponding discussion). However, the ability of SERS to detect individual miRNAs in mixed samples was not evaluated. For the current study, SERS spectra were collected for three miRNA samples, including synthetic let-7a, miR-133a, and a two-component mixture of 0.60 μg of let-7a and 0.40 μg of miR-133a. In this study, each sample was applied to three different SERS substrates, and five spectra were collected from each substrate. The instrument settings (e.g., microscope objective, laser power, and integration time) were optimized to maximize the signal-to-noise ratio without detector saturation. The average spectrum for each sample was calculated for each substrate where the spectra were baseline corrected and then unit vector normalized. FIG. 1 shows the overlaid spectra for each sample and each substrate. This plot reveals several significant findings. First, this plot shows that SERS spectra from miRNA are readily detectable utilizing the Ag nanorod array substrates as sensing platforms. The spectra shown are similar in the number and location of SERS bands, but notable differences in relative peak intensities and slight spectral shifts are observed. The spectra show spectral features in the range of 400-1800 cm⁻¹ that are consistent with published results (Kneipp, K.; Flemming, J. J. Mol. Struct. 1986, 145, 173-179; Nabiev, I. R.; Sokolov, K. V.; Voloshin, O. N. J. Raman Spectrosc. 1990, 21, 333-336; Otto, C.; Tweel, T. J. J. v.; deMul, F. F. M.; Greve, J. J. Raman Spectrosc. 1986, 17; Thornton, J.; Force, R. K. Appl. Spectrosc. 1991, 45, 1522-1526; Suh, J. S.; Moskovits, M. J. Am. Chem. Soc. 1986, 108, 4711-4718, which are herein incorporated by reference for the corresponding discussion). Relative band intensities at 650 cm⁻¹ (G in-phase ring stretching), 732 cm⁻¹ (A ring stretching), and 522 cm⁻¹ (G and A bending modes) are stronger for the let-7a sample than miR133a (Kneipp, K.; Flemming, J. J. Mol. Struct. 1986, 145, 173-179; Nabiev, I. R.; Sokolov, K. V.; Voloshin, O. N. J. Raman Spectrosc. 1990, 21, 333-336; Otto, C.; Tweel, T. J. J. v.; deMul, F. F. M.; Greve, J. J. Raman Spectrosc. 1986, 17, which are herein incorporated by reference for the corresponding discussion). Likewise, relative band intensities at 600 cm⁻¹, 794 cm⁻¹, 1306 cm⁻¹, and 1631 cm⁻¹ (C vibrational modes)^{38, 39} are stronger for the miR133a sample than let-7a (Nabiev, I. R.; Sokolov, K. V.; Voloshin, O. N. J. Raman Spectrosc. 1990, 21, 333-336; Otto, C.; Tweel, T. J. J. v.; deMul, F. F. M.; Greve, J. J. Raman Spectrosc. 1986, 17, which are herein incorporated by reference for the corresponding discussion). These results are readily explained by the fact that let-7a has a great A and G content than miR-133a while miR-133a has a greater C content. More details on correlating SERS spectra to miRNA sequence identity can be found in Driskell, J. D.; Seto, A. G.; Jones, L. P.; Jokela, S.; Dluhy, R. A.; Zhao, Y. P.; Tripp, R. A. Biosens. Bioelectron, which is incorporated herein by reference for the corresponding discussion).

The second significant finding from FIG. 1 is the high degree of spectral reproducibility apparent in the Figure. The spectra plotted in FIG. 1 reflect the average spectra acquired from three different substrates for each miRNA sample. Importantly, the relative intensities of the key bands indicative of each sample do not markedly differ from substrate-to-substrate. For example, as noted above, strong bands at 522 cm⁻¹, 650 cm⁻¹, and 732 cm⁻¹, and the weak bands at 600 cm⁻¹, 794 cm⁻¹, 1306 cm⁻¹, and 1631 cm⁻¹ are specific to let-7a. This same intensity pattern is obtained from each substrate. This level of spectral reproducibility suggests that calibration curves or multivariate regression models can be generated and used to test unknown samples for miRNA content.

The third important discovery from FIG. 1 is the finding that mixtures of miRNA sequences can be analyzed and discriminated. The sample containing 0.6 μg of let-7a and 0.4 μg of miR-133a results in a spectrum that is intermediate in relative intensities between let-7a and miR-133a. For example, the intensities of the bands located at 650 cm⁻¹, 732 cm⁻¹, 794 cm⁻¹, 1306 cm⁻¹, and 1631 cm⁻¹ are between the intensities of the let-7a and miR-133a samples. The SERS spectra of mixtures appear to be additive spectra of individual components, suggesting quantitative information regarding individual miRNA components in mixtures is possible.

To further explore the potential of SERS to extract quantitative information on individual miRNA components in mixtures, SERS spectra were collected for ten samples each including different concentrations of let-7a and miR-133a (Table 2). Average (n=15-30) spectra for five of the ten samples are presented in FIG. 2. As in FIG. 1, it is obvious that several bands track the relative concentrations of each component. The 650 cm⁻¹ and 732 cm⁻¹ bands increase in intensity as the concentration of let-7a increases, while the bands at 794 cm⁻¹, 1306 cm⁻¹, and 1631 cm⁻¹ increase in intensity as the miR-133a concentration increases. While these are not the only bands that correlate with miRNA concentrations, they are the most obvious based on visual inspection of the spectra.

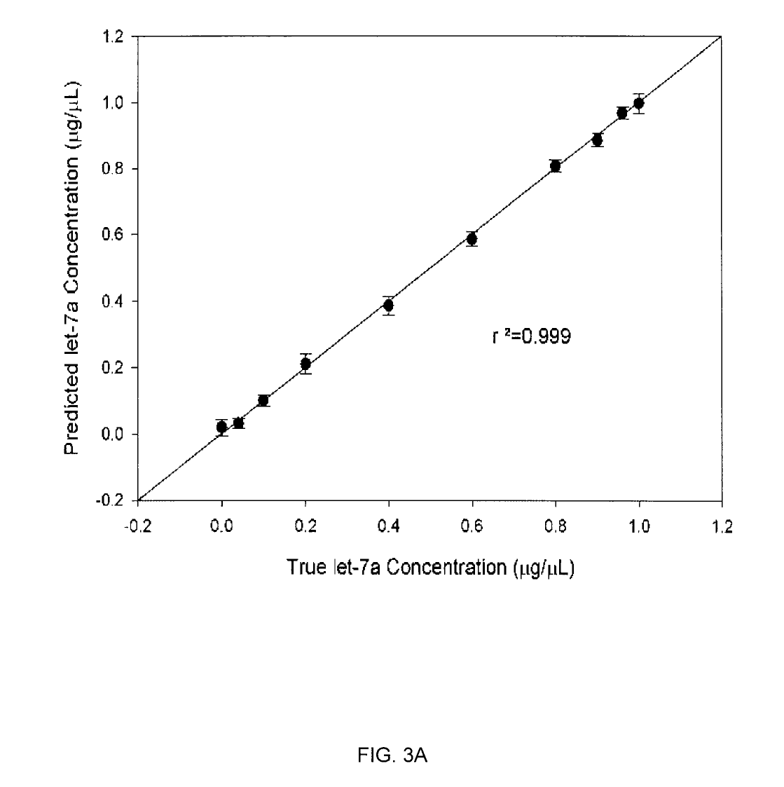

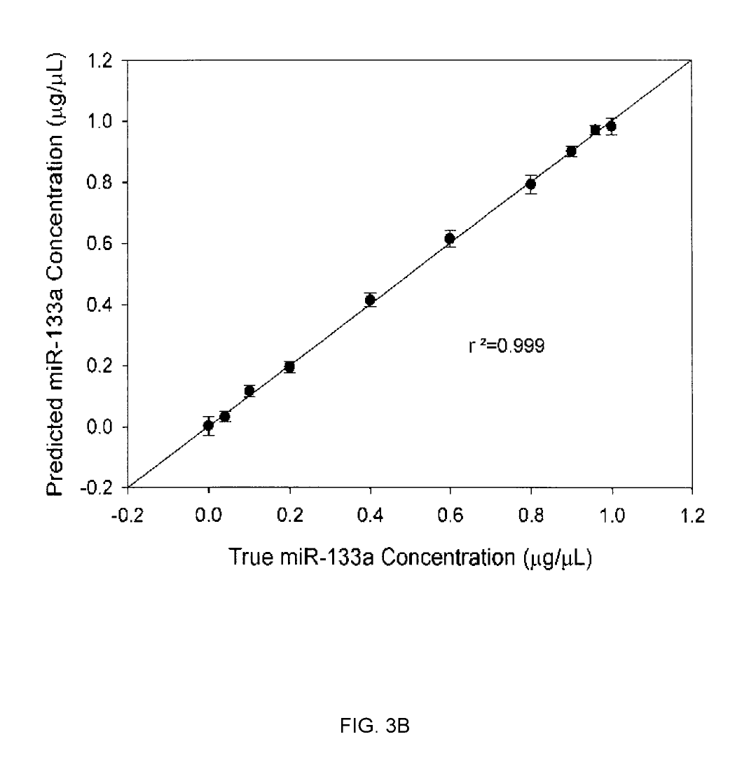

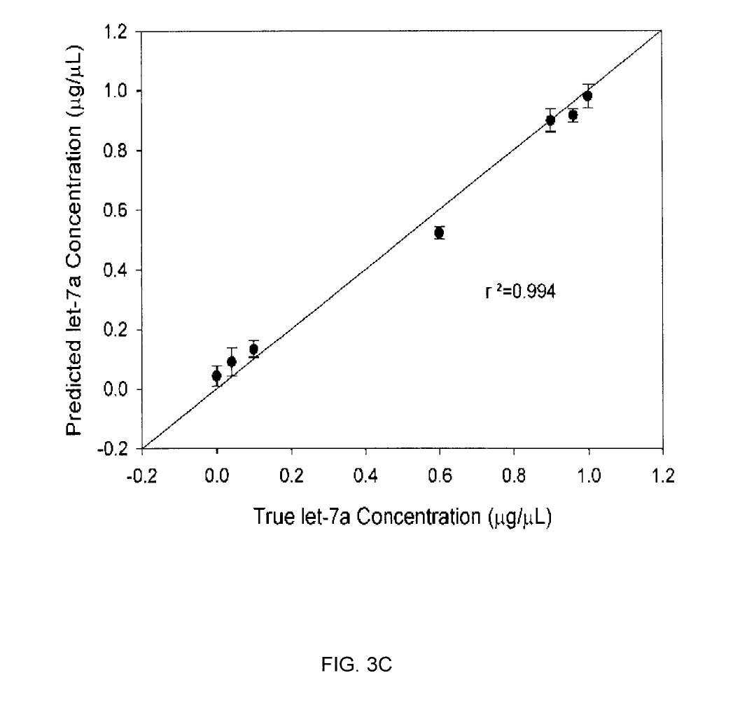

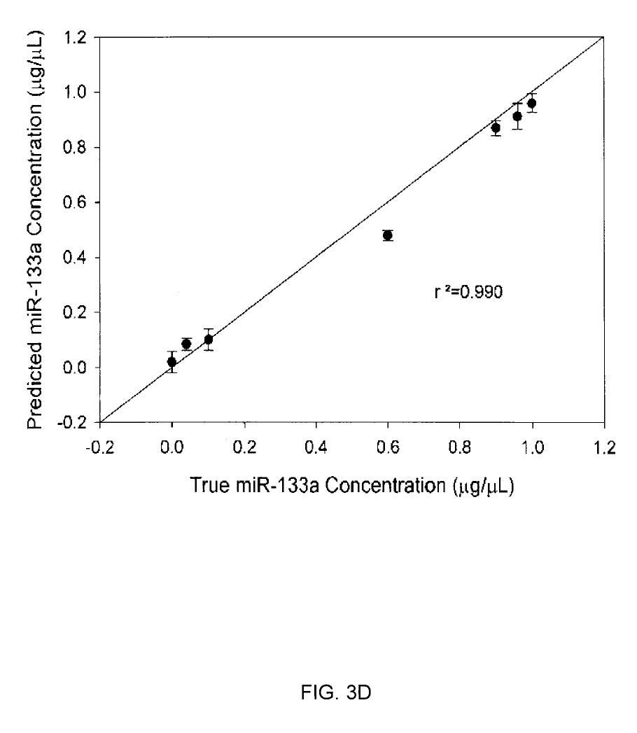

Partial least squares (PLS) regression methods were employed for quantitative analysis of let-7a and miR-133a concentrations in the two-component mixture. This type of multivariate calibration is more robust than univariate methods. The entire spectral range from 400-1800 cm⁻¹ was used to build PLS models for these mixtures. A PLS model was generated using the processed spectra (see Experimental Section) for each of the ten 2-component samples noted above. Spectra were collected from SERS substrates prepared in two different batches spanning three months. The root mean square error for cross validation (RMSECV) (leave-one-out) was analyzed to determine the optimum rank for the PLS model. As expected, the RMSECV rapidly drops with the initial factors, reaching a minimum value with the inclusion of 7 factors. Additional factors results in an increased RMSECV due to over-fitting of the data. Plots of the predicted concentrations from cross validation versus the true concentrations are displayed in FIGS. 3A and 3B. The model details are summarized in Table 3. This optimized model resulted in an RMSECV of 0.0262 μg/μL and an R² value of 0.999 for the prediction of both let-7a and miR-133a concentrations. The low value for RMSECV indicates a good fit of the data to the model.

TABLE 3
PLS regression model parameters and results for 2-component
mixtures. A leave-one-out algorithm was used for cross validation.

| | | let-7a | miR-133a |
|---|---|---|---|
| Cross validation | Concentration Range/μg/μL | 0.0-1.0 | 0.0-1.0 |
| | Spectroscopic Range/cm⁻¹ | 400-1800 | 400-1800 |
| | PLS Factors | 7 | 7 |
| | RMSECV/μg/μL | 0.0262 | 0.0262 |
| | R² | 0.996 | 0.996 |
| Test validation | Concentration Range/μg/μL | 0.0-1.0 | 0.0-1.0 |
| | Spectroscopic Range/cm⁻¹ | 400-1800 | 400-1800 |
| | PLS Factors | 9 | 9 |
| | RMSEP/μg/μL | 0.0544 | 0.0544 |
| | R² | 0.984 | 0.984 |

External validation of this PLS model for let-7a and miR-133a was accomplished with separate samples prepared on separate Ag nanorod substrates. Predicted versus true concentrations for the test data are shown in FIGS. 3C and 3D. The PLS regression model is summarized in Table 3. External validation resulted in a root mean square error of prediction (RMSEP) of 0.0544 μg/μL and R² values of 0.994 and 0.994 for the prediction versus true concentration curve for let-7a and miR-133a, respectively. These values of the RMSECV and RMSEP indicate that the selected rank does not result in over-modeling and that the model can be successfully applied to test unknown samples using SERS substrates prepared in future batches.

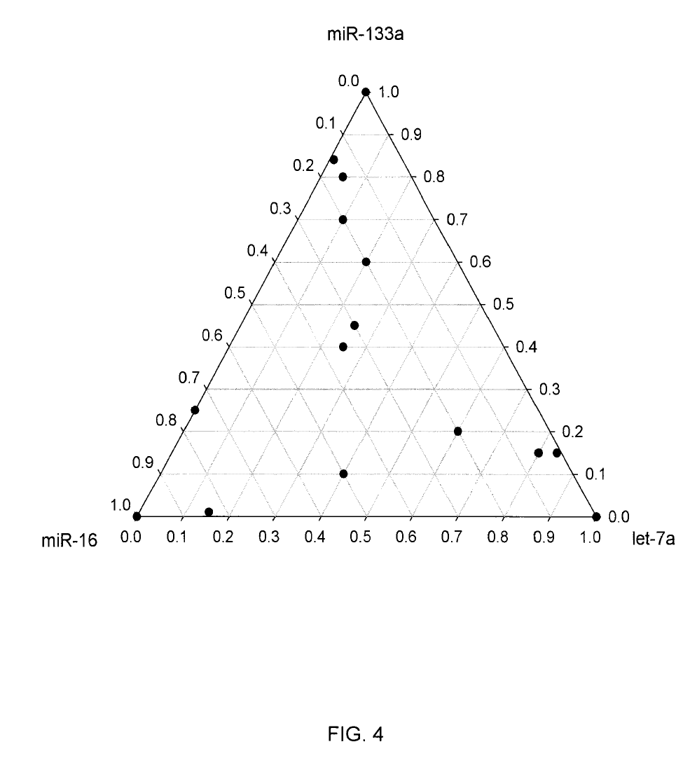

Quantitative Analysis of 3-Component Mixtures. In a second series of experiments, samples containing a mixture of three miRNAs were examined. These samples included varying concentrations of let-7a, miR-133a, and miR-16, while the total miRNA concentration was held constant at 1 μg/μL. This value was chosen since total RNA isolation that may be used for miRNA profiling often yields a total RNA concentration on the order of 1 μg/μL (Thomson, J. M.; Parker, J. S.; Hammond, S. M. In Methods in Enzymology; Rossi, J. J., Hannon, G. J., Eds.; Academic Press San Diego, Calif., 2007; Vol. Volume 427, pp 107-122, which is herein incorporated by reference for the corresponding discussion). FIG. 4 shows the composition of let-7a, miR-133a, and miR-16 concentrations. The samples were prepared to provide varying compositions ranging from individual miRNAs to several 3-component mixtures. The 3-component mixtures present a greater challenge for interpretation and quantification compared to the 2-component mixture. For example, when considering a 2-component mixture of let-7a and miR-133a, as the concentration of let-7a increases one expects the 731 cm⁻¹ band to increase in intensity. However, when considering a 3-component mixture, a similar increase in the intensity of 731 cm⁻¹ band could be the result of increasing the let-7a or miR-16 concentration. Thus, multivariate calibration, as opposed to univariate calibration, is used for the analysis of multi-component (n>2) mixtures, particularly when considering application of SERS miRNA profling to more than one miRNA, where typically miRNAs may be up- or down-regulated.

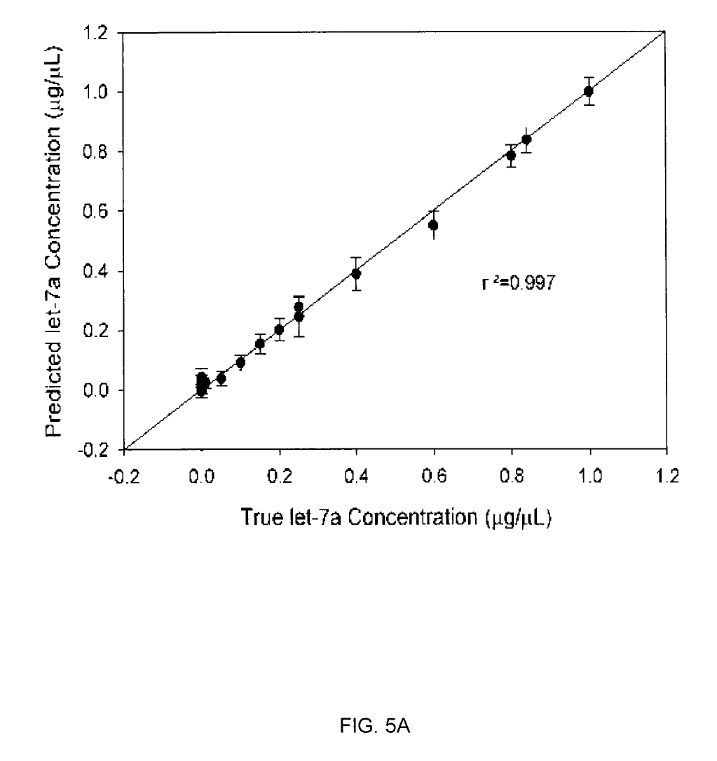

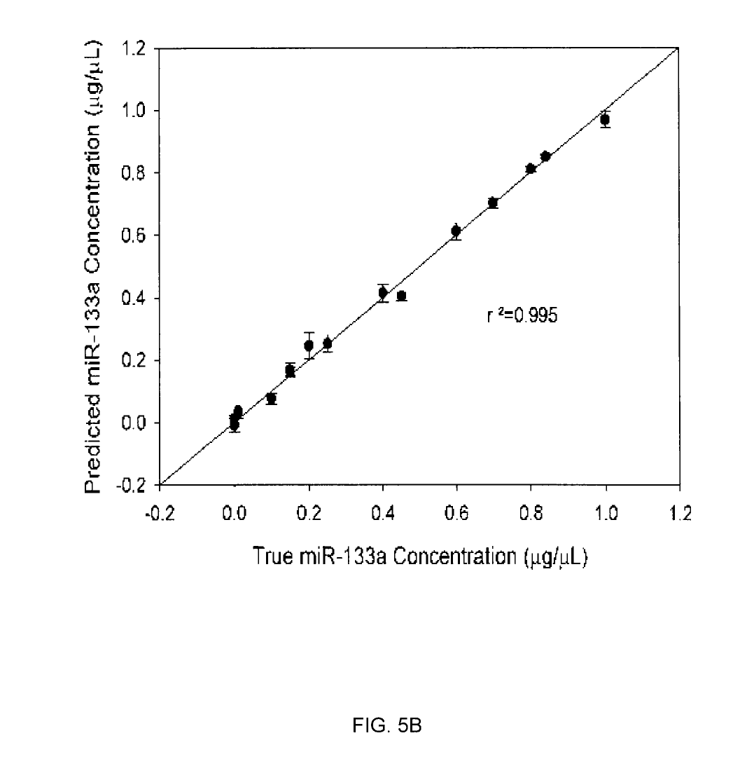

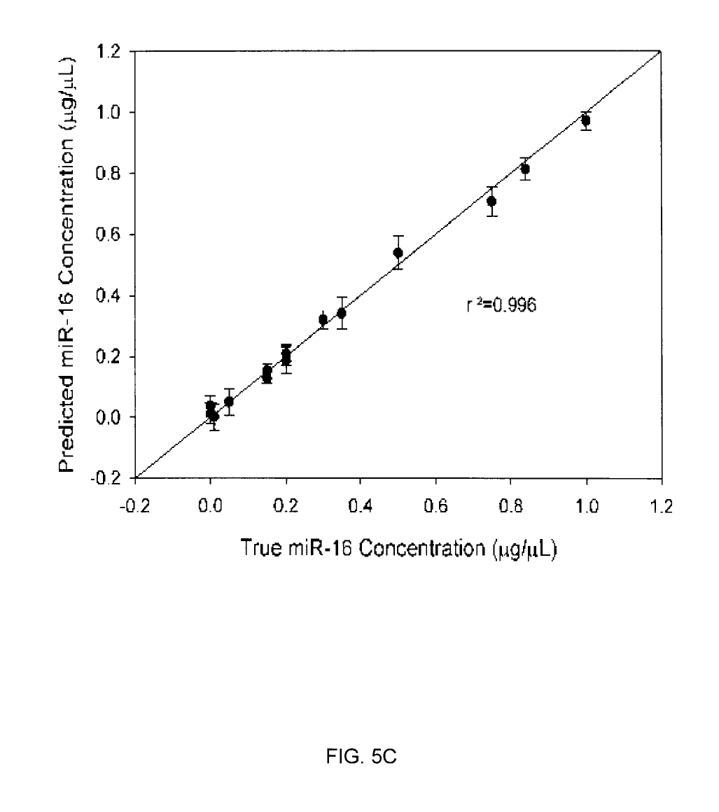

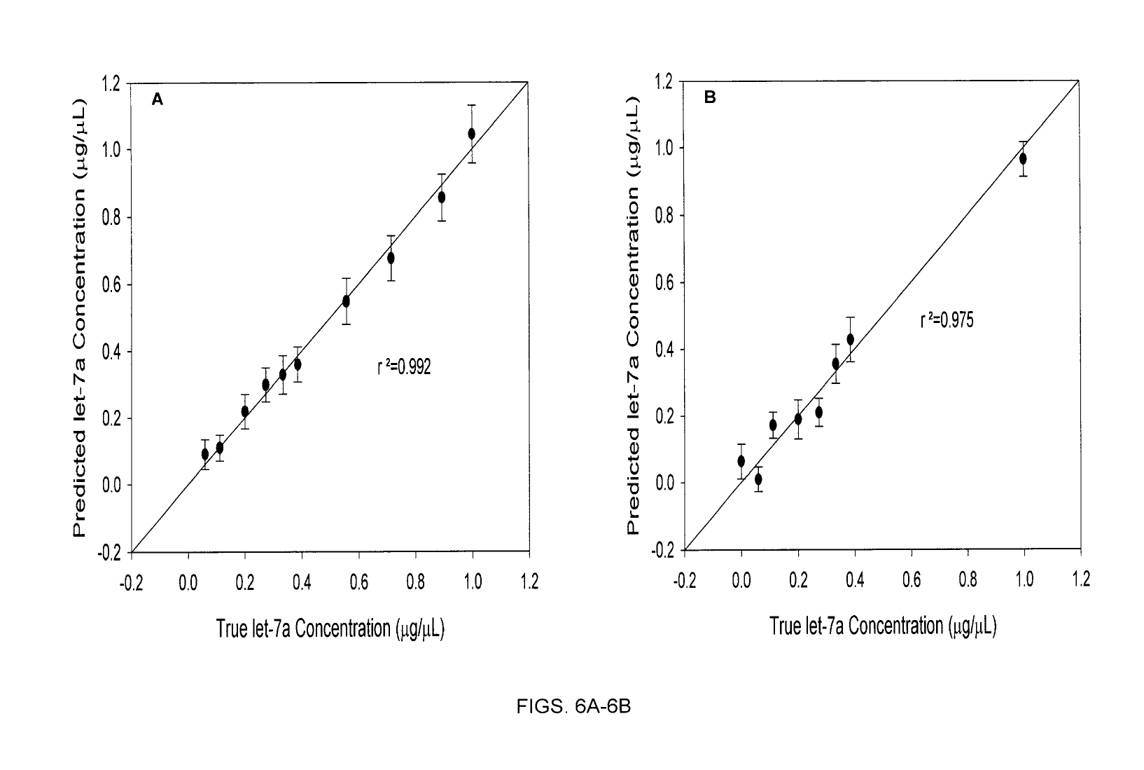

One microliter of each of the samples presented in FIG. 4 was applied to multiple SERS substrates (n=3-5), and five spectra were recorded from each substrate for a total of 15-25 spectra for each mixture. Venetian blinds cross validation was used to optimize the number of PLS factors in the model. The 250 spectra in the dataset were split into two subsets, one containing 90% of the data and the other 10% of the data. The larger subset was used to generate a model and predict the concentration of the smaller subset using different number of latent variables. The process was repeated nine times and the optimum number of PLS factors was determined by the number of latent variables which gave the smallest average prediction error sum of squares (PRESS). Nine factors were determined to be optimal. The cross validation results for the 3-component mixtures are plotted in FIG. 5. RMSECVs for let-7a, miR-133a, and miR-16 were calculated as 0.0460, 0.0340, and 0.0487 μg/μL, respectively, and each curve yielded an R² value greater than 0.995. Model details and results are summarized in Table 4. These RMSECV and R² values are evidence that the model is accurate and that multivariate analysis of SERS spectra can be used to successfully quantify each component in a tertiary mixture.

TABLE 4
PLS regression model parameters and results for 3-component
mixtures. A Venetian blinds algorithm with 10 splits was used
for cross validation.

| | | let-7a | miR-133a | miR-16 |
|---|---|---|---|---|
| Cross validation | Concentration | 0.0-1.0 | 0.0-1.0 | 0.0-1.0 |
| | Range/μg/μL | | | |
| | Spectroscopic | 400-1800 | 400-1800 | 400-1800 |
| | Range/cm⁻¹ | | | |
| | PLS Factors | 9 | 9 | 9 |
| | RMSECV/μg/μL | 0.046 | 0.34 | 0.0487 |
| | R² | 0.997 | 0.995 | 0.996 |

Quantitative Analysis of 5-Component Mixtures. Samples including five miRNAs, let-7a, miR-133a, miR-16, miR-21, and miR-24a, were prepared to emulate miRNA profiling. The goal of miRNA profiling studies is often to identify minor changes in the expression of one or a few miRNAs in the presence of a constant miRNA background. In this experiment, samples were prepared by varying let-7a concentrations while the total RNA concentration was held constant at 1 μg/μL by adjusting the concentration of the other four miRNAs. The relative ratios of the other four miRNAs were fixed to represent a constant background.

A PLS calibration model to predict the concentration of let-7a in these 5-component mixtures was generated from >250 spectra. Ten samples were prepared that spanned a concentration range of 0.050 μg/μL to 1.00 μg/μL for let-7a. More than 25 spectra were collected for each sample. Analysis of the RMSECV value for leave-one-out cross validation resulted in an optimum rank of 7 yielding a minimum RMSECV value of 0.0645 μg/μL. FIG. 5A shows the correlation between the cross validation predictions for let-7a concentrations and the true let-7a concentrations.

To more completely test the robustness of SERS miRNA profiling, additional let-7a mixtures were prepared and applied to Ag nanorod SERS substrates fabricated independently of those used to build the calibration model. This test set of data was used to externally validate the calibration model. The root mean square error of prediction (RMSEP) was used as a criterion to judge the model performance. Test spectra (n=96) were acquired for 8 different samples and the PLS model was used to predict the let-7a concentration. A plot of the predicted let-7a concentrations versus the true let-7a concentrations is presented in FIG. 5B. The figure reveals good agreement between the predicted concentrations and the true concentration, with a RMSEP of 0.0684 μg/μL. The low value for RMSEP indicates a good fit of the model to the data. The close match of the RMSEP to the RMSECV reveals the model was not over-fitted to the calibration dataset. The PLS model and results for the 5-component experiments are detailed in Table 5

TABLE 5
PLS regression model parameters and results for the quantification
of let-7a in 5-component mixtures. A leave-one-out algorithm was
used for cross validation.

| | | let-7a |
|---|---|---|
| Cross validation | Concentration Range/μg/μL | 0.0-1.0 |
| | Spectroscopic Range/cm⁻¹ | 400-1800 |
| | PLS Factors | 7 |
| | RMSECV/μg/μL | 0.0645 |
| | R² | 0.992 |
| Test validation | Concentration Range/μg/μL | 0.0-1.0 |
| | Spectroscopic Range/cm⁻¹ | 400-1800 |
| | PLS Factors | 7 |
| | RMSEP/μg/μL | 0.684 |
| | R² | 0.975 |

A closer examination of these results in light of the experimental design underscores the sensitivity of this method. The results from the 5-component mixture studies show that 0.05 μg (˜6 picomoles) of let-7a is detectable in the presence of a miRNA background. However, current experimental methods allow the sample to spread evenly over a 3 mm² area of the Ag nanorod array. In addition, the Raman excitation laser is only exciting an area of ˜1200 μm². Therefore, less than 0.05%, or ˜3 femtomoles, of the miRNA in the sample is producing the measured signal. Opportunities for improvement of SERS sensitivity of miRNA detection include engineering of the SERS substrate to confine the sample within the focal diameter of the laser spot. Also, changes in the Raman microscope configuration may lead to improvement in collection efficiency by a factor of ˜100.

It should be noted that the concentration of the samples in the PLS training set greatly affects the limit of detection. Ideally, one would select training samples spanning the concentration range of interest. Therefore, using lower concentrations to train the PLS model may lead to even lower detection limits. Taken together, implementation of these changes to both the instrumental parameters and statistical model suggests that less than 30 attomoles of miRNA could readily be detected using Ag nanorod-based SERS without any amplification steps.

### CONCLUSIONS

The experiments reported here demonstrate the utility of SERS for the rapid (10 s), sensitive, and accurate detection and quantitative analysis of individual miRNA sequences in multicomponent mixtures. These studies indicate that SERS can be used as a label-free method to detect miRNAs, and suggest that SERS may provide a novel platform technology to identify miRNA profiles important in gene regulation and disease pathogenesis. Conventional miRNA detection methods, e.g., northern blotting, PCR and microarray hybridization, have provided foundational evidence for many important roles of miRNAs. Unfortunately, all these methods are limited in their ability to detect miRNAs. The limitations of these methods include, i) their sensitivity is limited to efficient and specific hybridization; ii) the assays require relatively large sample concentrations; and ii) the methods are labor and time intensive. The SERS methodology described in this study overcomes many of these limitations by i) providing rapid (10 s) and quantitative detection and analysis of minimal sample concentrations, ii) by eliminating the need for fluorescent probe labeling, and ii) by eliminating the hybridization steps required for amplification of the analyte. The studies presented here indicate that at least two approaches for SERS-based miRNA profiling may be pursued. The first would follow a similar procedure to that reported here where total RNA or purified small RNA extracted from cells or tissue could be applied to a SERS substrate and analyzed using PLS regression models for each suspected miRNA. Benefits of this approach include minimal sample preparation, no labeling, extremely short analysis time, and no hybridization step. Evidence for this approach lays in the successful analysis of 3- and 5-component miRNA mixtures.

A second conceptual approach parallels that of a microarray. In this format, probe sequences complimentary to targeted miRNA could be immobilized on the SERS substrate in an array format using established immobilization chemistry. Hybridization of miRNA to the probes could be directly detected via SERS without the need for a label. Excellent accuracy in the quantification of the 2-component mixtures above provides evidence in support of this approach. Selective binding of miRNA allows the background total RNA to be removed, eliminating challenges associated with background signals. Furthermore, micro-printing techniques that facilitate confinement of the target miRNA to a small area on the substrate of approximately the same size of the laser spot would enhance detection. While this format would be subject to the same non-specific binding limitation of current microarray methods, the chemically-sensitive SERS signature is capable of discriminating against mismatched hybridization (Driskell, J. D.; Seto, A. G.; Jones, L. P.; Jokela, S.; Dluhy, R. A.; Zhao, Y. P.; Tripp, R. A. Biosens. Bioelectron, which is herein incorporated by reference for the corresponding discussion). Moreover, a SERS-based readout of microarray hybridization does not require the additional time and cost of labeling with fluorophores and is not hindered by the lack of standardized normalization methods.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In embodiments where “about” modifies 0 (zero), the term “about” can include ±1%, ±2%, ±3%, ±4%, ±5%, ±6%, ±7%, ±8%, ±9%, ±10%, or more of 0.00001 to 1. In addition, the phrase “about to ‘y’” includes “about ‘x’ to about ‘y’”.

It should be emphasized that the above-described embodiments of the present disclosure are merely possible examples of implementations, and are merely set forth for a clear understanding of the principles of the disclosure. Many variations and modifications may be made to the above-described embodiments. All such modifications and variations are intended to be included herein within the scope of this disclosure and protected by the following claims.

Therefore, at least the following is claimed:

1. A method for analysis of individual components in a multicomponent sample, comprising:
applying the multicomponent sample to a surface enhanced Raman spectroscopy (SERS) platform;
obtaining a unique SERS spectrum for each component of the multicomponent sample;
analyzing the unique SERS spectrum of each component of the multicomponent sample; and
determining a disease or condition based on an identity of at least one individual component.

2. The method of claim 1, wherein the individual components of the multicomponent sample comprise individual miRNA or nucleotide sequences.

3. The method of claim 1, wherein the SERS platform comprises a Ag nanorod array substrate.

4. The method of claim 3, wherein the Ag nanorod array substrate is prepared by oblique angle vapor deposition (OAD).

5. The method of claim 1, wherein the unique SERS spectra of each component of the multicomponent sample are analyzed using partial least squares (PLS) regression analysis.

6. A method for identification, differentiation, and/or quantification of individual components in a multicomponent sample, comprising:
applying the multicomponent sample to a surface enhanced Raman spectroscopy (SERS) platform;
obtaining a unique SERS spectrum for each component of the multicomponent sample; and
analyzing the unique SERS spectrum of each component of the multicomponent sample.

7. The method of claim 6, wherein the individual components of the multicomponent sample comprise individual miRNA or nucleotide sequences.

8. The method of claim 7, wherein the method is used for miRNA profiling.

9. The method of claim 6, wherein the SERS platform comprises a Ag nanorod array substrate.

10. The method of claim 9, wherein the Ag nanorod array substrate is prepared by oblique angle vapor deposition (OAD).

11. The method of claim 6, wherein the unique SERS spectra of each component of the multicomponent sample are analyzed using partial least squares (PLS) regression analysis.

12. The method of claim 6, wherein the multicomponent sample comprises 2 components.

13. The method of claim 12, wherein the 2 components are miRNA selected from the group consisting of: hsa-let-7a, hsa-miR-133a, hsa-miR-21, hsa-miR-16, and hsa-miR-24a.

14. The method of claim 6, wherein the multicomponent sample comprises 3 components.

15. The method of claim 14, wherein the 3 components are miRNA selected from the group consisting of: hsa-let-7a, hsa-miR-133a, hsa-miR-21, hsa-miR-16, and hsa-miR-24a.

16. The method of claim 6, wherein the multicomponent sample comprises 5 components.

17. The method of claim 16, wherein the 5 components are miRNA selected from the group consisting of: hsa-let-7a, hsa-miR-133a, hsa-miR-21, hsa-miR-16, and hsa-miR-24a.

18. The method of claim 7, wherein the individual miRNA and/or nucleotide sequences can be detected in about 10 to 30 seconds.

19. The method of claim 10, wherein the Ag nanorod array substrate comprises individual nanorods with a length of about 900 nm.

20. The method of claim 6, wherein a multicomponent sample concentration is dilute.

21. The method of claim 7, wherein the multicomponent sample concentration is about 0.04 to 1.0 μg/μL for each miRNA in the sample.

22. The method of claim 9, wherein the nanorods are selected from one of the following materials: a metal, a metal oxide, a metal nitride, a metal oxynitride, a polymer, a multicomponent material, and a combination thereof.

23. The method of claim 22, wherein the material is selected from one of the following: silver, nickel, aluminum, silicon, gold, platinum, palladium, titanium, cobalt, copper, zinc, oxides of each, nitrides of each, oxynitrides of each, carbides of each, and combinations thereof.

24. The method of claim 6, wherein the multicomponent sample is selected from the group consisting of: blood, saliva, tears, phlegm, sweat, urine, plasma, lymph, spinal fluid, cells, microorganisms, a combination thereof, and aqueous dilutions thereof.

25. The method of claim 7, wherein the identification of the individual miRNA is an indicator for the detection of cancer.

26. A method for quantification of individual components in a multicomponent sample, wherein the individual components of the multicomponent sample comprise individual miRNA sequences, comprising:
applying the multicomponent sample to a surface enhanced Raman spectroscopy (SERS) platform;
obtaining a unique SERS spectrum for each of the individual miRNA sequences in the multicomponent sample; and
analyzing the unique SERS spectra using partial least squares (PLS) regression analysis.


SEQUENCE LISTING (5 sequences)

SEQ ID NO: 1 - RNA, 22 nt, Artificial Sequence
  Chemically synthesized
uagcagcacguaaauauuggcg

SEQ ID NO: 2 - RNA, 22 nt, Artificial Sequence
  Chemically synthesized
uagcuuaucagacugauguuga

SEQ ID NO: 3 - RNA, 22 nt, Artificial Sequence
  Chemically synthesized
uggcucaguucagcaggaacag

SEQ ID NO: 4 - RNA, 22 nt, Artificial Sequence
  Chemically synthesized
uugguccccuucaaccagcugu

SEQ ID NO: 5 - RNA, 22 nt, Artificial Sequence
  Chemically synthesized
ugagguaguagguuguauaguu